(19) United States
(12) Patent Application Publication
(10) Pub. No.: US 2010/0105144 A1
(43) Pub. Date: Apr. 29, 2010

(54) ONIUM SALTS AND USE THEREOF FOR DETECTING AND ASSAYING METALS

(75) Inventors: Gilles MARCHAND, Pierre Chatel (FR); Faidjiba LOE MIE, Grenoble (FR); Mathieu PUCHEAULT, Chateaubourg (FR); Michel VAULTIER, Chateaugiron (FR); Mireille BLANCHARD-DESCE, Rennes (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: COMMISSARIAT A L'ENERGIE ATOMIQUE, Paris (FR)

(21) Appl. No.: 12/427,320

(22) Filed: Apr. 21, 2009

(30) Foreign Application Priority Data
Apr. 21, 2008 (FR) 08 02209

(51) Int. Cl. G01N 33/20; C07D 215/14; C22B 1/00

(52) U.S. Cl. 436/73; 546/174; 75/711

(57) ABSTRACT

Task-specific onium salts, process for preparing them and use thereof for detecting and assaying metals, in particular heavy metals, in an aqueous medium.

The invention relates to novel molecules of the task-specific onium salt type, to a process for preparing them and to their use for detecting and assaying metals, in particular heavy metals, in an aqueous medium.

The assaying of heavy metals, for instance Hg, Pb or Cd, in water is a health and environmental necessity. The reason for this is that, of the 41 elements corresponding to this definition, 21 elements are toxic to man and to the environment.

According to the processes commonly used, the assaying of heavy metals in an aqueous phase may be performed according to two methods:

Direct detection in the aqueous phase: This assay method involves techniques such as ICP/MS (inductively-coupled plasma mass spectrometry) or AAS (atomic absorption spectroscopy). Although very sensitive, these techniques are laborious and difficult to integrate into a portable system. Analysis of the elements thus takes place off-site rather than in the field. The known portable systems involve an electrochemical detection (polarography, CV (cyclic voltammetry) or ASV (anodic stripping voltammetry), for instance the Trace Detect® machine sold by the company Fondis Electronic. These techniques are limited by the fact that certain metals have no electrochemical signature in the electrochemical window of water (Al, Ti, Li, etc.) or are not electrodeposable on an electrode (ASV). Furthermore, the selectivity of the measuring method (in this case, it is the measuring method that provides the selectivity) turns out to be occasionally low in media containing several metals that have similar electrochemical responses. Finally, the reliability of such measurements is limited especially in terms of reproducibility for compounds at low concentrations. Another limitation of ASV lies in the fact that this technique is too slow for detecting extremely rapid transient pollution events (accumulation on an electrode is necessary for stripping) whereas an optical measurement allows a real-time measurement. Portable lead detectors using X-rays as the detection technique also exist. The X-ray source excites the electrons of the deep shells (K or L) of heavy metals. These metals relax this excitation by emitting X radiation at energies that are characteristic for the metal. The measurements are rapid and the sensitivity is good, and compatible with the maximum admissible limits in force. However, one problem associated with this type of detector lies in the hazard involved in handling these X-ray emission tubes.

Whatever the method used, the direct assaying of metals in an aqueous medium outside a laboratory, especially in a natural environment, often proves to be very problematic on account of the presence of these elements in trace form and of the limitations of the current techniques in terms of sensitivity. Specifically, the known on-line measuring devices reach a detection threshold of a few micrograms per litre, whereas the objectives of the directive 2006/11/EC are of the order of a few thousandths of a microgram.

TABLE 1
evaluation of the chemical state of waters:
threshold values (μg/L)

| | | Interim threshold | |
|---|---|---|---|
| | PNEC* for | values of the satisfactory | Threshold values** |
| | freshwater | chemical | delimiting waters |
| | organisms | state (Directive | of very good quality |
| | (INERIS, 2007) | 2005/14/EC) | (QES***-water) |
| Cd | 0.21 | 5 | 0.004 |
| Cu | 1.6 | — | 0.1 |
| Hg | 0.24 | 1 | 0.007 |
| Pb | 5 | bn + 0.4 | 0.52 |
| Zn | 8.6 | — | 0.43 |

*predictable no-effect concentrations
**for a raw water of moderate hardness
***QES: Quality Evaluation System for water courses
bn: background noise of the assay.

Thus, to improve the accuracy and sensitivity and/or the detection threshold of their assay, a phase of extraction in small volumes of organic solvents, in order to partially purify and concentrate the compounds to be assayed, proves to be necessary.

Detection after concentration by liquid/liquid extraction: the liquid/liquid extraction of metals from an aqueous phase with an organic phase makes it possible to achieve concentration of the metals and to reach or lower, for sparingly concentrated samples, the detection threshold of the chosen detection method. The efficient extraction of metals, from the aqueous medium to the organic medium, can be improved by using chelating molecules, dissolved in the organic solvent. The resulting complex has greater affinity for the receiving phase than the metal ion alone. This extraction may be selective for a given metal, or unselective. After extraction, the assay is performed by the techniques mentioned previously (ICP/MS, AAS, electrochemistry) with the same problems in terms of bulk. Occasionally also, the complexing agent is fluorescent and the complexation of the metal results in a change in the emission wavelength of the complexing agent (Tetrahedron Letters Vol. 48, Issue 37, 10 Sep. 2007, pp. 6527-6530), but the known processes involving this method have several drawbacks: the detection limit is hampered by the offset phenomenon (partial superposition of the peak produced by the complexing agent alone and of the peak produced by the complexing agent bound to the metal), sets of filters are necessary. Irrespective of the chelating agent used, the ionic complex (chelating agent+ion) must be very sparingly soluble in water, otherwise it remains partially dissolved in the aqueous phase, the extraction is inefficient and the assay is biased.

With standard solvents, in the majority of cases, the extraction yields are low since the extraction is thermodynamically unfavoured (S. Dai et al., J. Chem. Soc. Dalton Trans., 1999, 1201-1202). Furthermore, the volatility of these solvents (VOS, volatile organic solvents) makes them difficult to use in very small volume without the use of cowling (for instance a channel), which makes the device more complex (Tokeshi M. et al., Anal. Chem. 2002, 1565-1571) and the implementation of the processes more difficult. In particular, the treatment of a large number of samples in parallel proves to be complicated. In order to overcome these problems, it has been proposed to use ionic liquids (WO 2004/005222). The reason for this is that ionic liquids have noteworthy properties: i) they are non-volatile solvents (their vapour pressure is negligible) and they can thus be used in very small volume; ii) they can be designed to be immiscible with water as a function of the anion or cation used and they are capable of dissolving the majority of organic and mineral molecules; iii) their partition coefficients (Nernst distribution coefficient) are often high. Thus, tests of extraction of Cs⁺ ions in these solvents containing chelating agents such as crown ethers (Luo H. et al., Anal. Chem. 2004, 3078-3083) showed an increase in extraction efficacy when compared with the solvents usually used (chloroform). Document DE3607982 describes the extraction of cations and anions using onium salts. However, the compounds described in the said document do not enable direct assaying of the detected ions.

The document Kumano et al., Int. J. Modern Physics, 20 (25, 26, 27), 2006, 4051-4056 describes the use of 8-hydroxyquinoline as chelating agent for extracting metals. However, this process does not make it possible to perform direct detection of the detected species.

However, extraction with ionic liquids in the presence of a chelating molecule also has drawbacks: i) once the ionic complex (chelating agent+ion) has been formed, it may possibly return into the aqueous phase since the ion often remains hydrated, and the extraction is then less efficient; ii) the detection and assaying of the metal are performed via the techniques described previously, with the same drawbacks. Although the first of these problems has been solved by supporting the chelating functions on ionic liquids in order to form task-specific ionic liquids (TSIL) (Visser et al., Chem. Commun., 2001, 135-136; Environ. Sci. Technol., 2002, 36, 2523-2529), the second problem remains. In the latter publication, the detection is performed by measuring the radioactivity (²⁰³HgCl₂ and ¹⁰⁹CdCl₂), this system being even more laborious than those mentioned previously.

The invention has the advantage of solving all these problems by proposing novel molecules that are able simultaneously to perform very efficient extraction of metals from an aqueous medium, and to perform a very sensitive detection and possible assay thereof by using techniques that are not hazardous to the operative or those in his vicinity. These molecules, of the hydrophobic task-specific onium salt type, comprising a group from the quinoline family, can simultaneously scavenge metals in an aqueous medium, extract them therefrom and emit a fluorescence signal that is a consequence of this scavenging.

A first subject of the invention is molecules of formula (I) below:

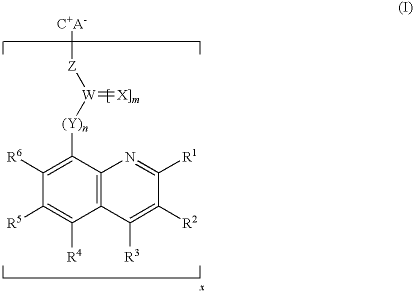

in which:

X represents a group chosen from: O, NR, S, PR, Se; and R represents a group chosen from: H, a C₁-C₁₂ alkyl group optionally comprising one or more heteroatoms, a C₂-C₁₂ alkenyl group optionally comprising one or more heteroatoms, a C₆-C₁₂ aryl group optionally comprising one or more heteroatoms, a C₆-C₁₂ aralkyl group optionally comprising one or more heteroatoms;

Y represents a group chosen from: O, S, NR′, PR′, CR′R″, a C₁-C₁₂ alkanediyl group optionally comprising one or more heteroatoms, a C₂-C₁₂ alkenediyl group optionally comprising one or more heteroatoms, a C₆-C₁₂ aryl group optionally comprising one or more heteroatoms, a C₆-C₁₂ aralkanediyl group optionally comprising one or more heteroatoms; and R′ and R″, which may be identical or different, represent a group chosen from: H, a C₁-C₁₂ alkyl group optionally comprising one or more heteroatoms, a C₂-C₁₂ alkenyl group optionally comprising one or more heteroatoms, a C₆-C₁₂ aralkyl group optionally comprising one or more heteroatoms, a C₆-C₁₂ aryl group optionally comprising one more heteroatoms;

n is an integer chosen from: 0, 1;

m is an integer chosen from: 1, 2;

x is an integer chosen from: 1 and 2;

W represents an atom chosen from: C and S;

Z represents a group chosen from: a C₁-C₁₂ alkanediyl group optionally comprising one or more heteroelements, a C₂-C₁₂ alkenediyl group optionally comprising one or more heteroelements, a C₆-C₂₀ aralkanediyl group optionally comprising one or more heteroelements, a C₆-C₁₂ aryl group optionally comprising one or more heteroelements, the heteroelement(s) being chosen from: Cl, Br, O, S, NR″′, PR″′; and R″′ represents a group chosen from: H, a C₁-C₁₂ alkyl group, a C₂-C₁₂ alkenyl group, a C₆-C₁₂ aryl group and a C₆-C₁₂ aralkyl group;

R¹, R², R³, R⁴, R⁵ and R⁶, which may be identical or different, each represent a group chosen from the following list: H, Cl, Br, a C₁-C₁₂ alkyl group optionally comprising one or more heteroelements, a C₂-C₁₂ alkenyl group optionally comprising one or more heteroelements, a C₆-C₁₂ aryl group optionally comprising one or more heteroelements, a C₆-C₁₂ aralkyl group optionally comprising one or more heteroelements, OH, a group O⁷, NH₂, a group NR⁷R⁸, SH, a group SR⁷; and R⁷ and R⁸ are chosen, independently of each other, from the following list: a C₁-C₁₂ alkyl group, a C₂-C₁₂ alkenyl group, a C₆-C₁₂ aryl group, a C₆-C₁₂ aralkyl group; the heteroelement(s) being chosen from: Cl, Br, O, S, NR″′, PR″′; and R″′ represents a group chosen from: H, a C₁-C₁₂ alkyl group, a C₂-C₁₂ alkenyl group, a C₆-C₁₂ aryl group and a C₆-C₁₂ aralkyl group.

→C⁺A⁻ represents a task-specific onium salt.

When x=1, C⁺A⁻ may be bonded to Z either via the anion A⁻ or via the cation C⁺.

When x=2, the anion A⁻ and the cation C⁺ are each bonded to a group Z, the two groups

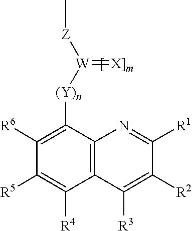

being identical or different.

The expression “onium salt” denotes ammonium, phosphonium or sulfonium salts and also any salt resulting from the quaternization of an amine, a phosphine, a thioether or a heterocycle containing one or more heteroatoms such as S, N or P. The term “task-specific onium salt” means an onium salt in which either the cation or the anion bears a function that is capable of complexing a metal (depending on the case, they are referred to as a “functional cation” and/or a “functional anion”). The anion and the cation may be simultaneously functional, each being bonded to an identical or different group Z.

C⁺ represents a functional or non-functional cation. C⁺ is preferentially chosen from ammonium ions, but it may also be chosen, for example, from: a pyrrolidinium, a phosphonium, an imidazolium, a pyridinium, a piperidinium or a guanidinium.

A⁻ represents a functional or non-functional anion. A⁻ may be chosen, for example, from the following ions: hexafluorophosphate (PF₆⁻), bis(trifluoromethanesulfon-amide) (NTf₂⁻), a tris(perfluoroalkyl)trifluorophosphate (FAP) ion such as tris(pentafluoroethyl)trifluoro-phosphate (FAP), triflate (TfO⁻), nonaflate (NfO⁻), or any other hydrophobic anion, the tetrafluoroborate ion BF₄⁻ or the chloride ion Cl⁻.

According to one preferred variant of the invention, the variables C⁺, A⁻, X, Y, W, R¹, R², R³, R⁴, R⁵ and R⁶ are chosen such that the compound of formula (I) is hydrophobic. C⁺ and A⁻ are advantageously chosen such that the compound of formula (I) is not water-soluble. Thus, for example, if the cation is functional, the anion is preferably chosen from hydrophobic anions, for instance a hexafluorophosphate ion (PF₆⁻) or a bis(t/ifluoromethanesulfonamide) ion (NTf₂⁻).

Preferably, in formula (I), one or more of the following conditions are satisfied:

X represents a group chosen from: O, S; even more preferentially, X represents O,

Y represents a group chosen from: O, S, NH, a C₁-C₆ alkanediyl group; even more preferentially, Y represents O,

n=1,

m=1,

x=1,

W represents a carbon atom: C,

Z represents a group chosen from: a C₁-C₆ alkanediyl group optionally comprising one or more heteroelements, a C₆-C₂₀ aralkanediyl group optionally comprising one or more heteroelements, the heteroelement(s) being chosen from: O, S, NH; preferably, Z is chosen from C₁-C₆ alkanediyl groups optionally comprising one or more oxygen atoms in or at the end of the alkyl chain, and C₆-C₂₀ aralkanediyl groups optionally comprising one or more oxygen atoms,

R¹, R², R³, R⁴, R⁵ and R⁶, which may be identical or different, each represent a group chosen from the following list: H, a C₁-C₆ alkyl group, even more preferentially from H and a C₁-C₃ alkyl, and advantageously from H and CH₃,

C⁺ represents a group

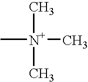

A⁻ represents a group chosen from: PF₆⁻ (hexafluorophosphate), NTf₂⁻ (bis(trifluoromethanesulfonamide)), tris(pentafluoroethyl)trifluorophosphate; advantageously, A⁻ represents NTf₂⁻.

The following molecules are more particularly a preferred subject of the invention:

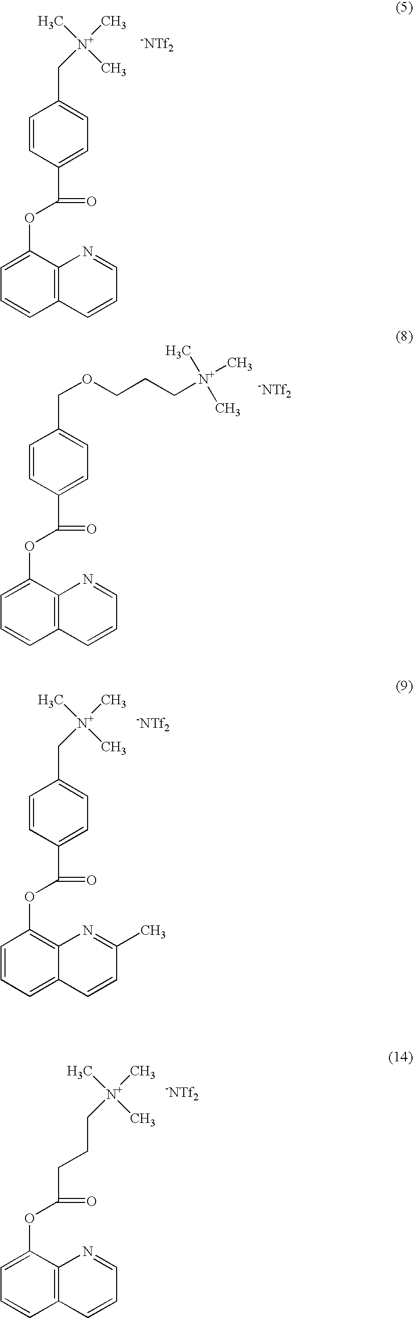

The task-specific onium salt (I) has the following advantages:

since the substituted quinoline function, which is capable of complexing the metal, is borne by a hydrophobic onium salt, the risks of diffusion of the (I)-metal ion complex into the aqueous phase are very limited;

the quinoline group performs both the chelation and the detection via fluorescence emission, thus enabling direct detection of the metal by a suitable fluorescence device.

It is a system that functions in the following manner: the quinoline fluoresces sparingly or not at all in the absence of heavy metals in the medium, and then, when there is complexation between the metal and (I), fluorescence emission then takes place.

The synthesis of the compounds of formula (I) is performed with the aid of organic synthesis methods that are well known to those skilled in the art, as is illustrated for certain compounds below in the examples.

The task-specific onium salts (I) may be used neat if they are liquid, or optionally dissolved in a solvent, whether these salts are in solid, liquid or oily form. When they are used in solution, they are preferably dissolved in a solvent that is hydrophobic or sparingly miscible with the aqueous phase containing the ion to be extracted. For example, they may be in solution in a halogenated solvent such as chloroform, dichloromethane, dichloroethane or CCl₄, a fluorinated solvent, in ethyl acetate, oxazole or mesityl oxide. Advantageously, the compounds of formula (I) are dissolved in a hydrophobic room-temperature ionic liquid (RTIL) matrix.

The term “ionic liquid” denotes a salt or a mixture of salts whose melting point is between −100° C. and 250° C. The term “ionic liquid matrix” means a functionalized or non-functionalized ionic liquid, which is liquid at room temperature, capable of dissolving one or more chemical species such as mineral or organic salts (such as task-specific onium salts), organic molecules and polymers of natural or synthetic origin. This ionic liquid matrix may be constituted of a pure ionic liquid or may be a mixture of several ionic liquids, which are functionalized or non-functionalized, and it may also be constituted of a mixture of one or more hydrophobic solvents as listed above with one or more ionic liquids. Among the most commonly known ionic liquids, mention may be made of N,N-dialkylimidazolium, N-alkylpyridinium, N,N-dialkylpyrrolidinium, N,N-dialkylpiperidinium and N,N-trialkylammonium agents (Wasserscheid P. et al., Ionic liquids in synthesis, VCH: Weinheim, 2003; Wasserscheid P. et al., Chem. Rev. 1999, 99, 2071; Rogers R. D., Seddon K. R. ACS Symposium Series 818, American Chemical Society, Washington DC, 2002; Rogers R. D., Seddon K. R. ACS Symposium Series 856, American Chemical Society, Washington DC, 2003; Bates E. D., J. Am. Chem. Soc., 2002, 124, 926). Advantageously, an ionic liquid chosen from those not having an aromatic backbone and whose absorbance has little or no negative impact on the excitation or emission of the molecules (I) is used, for instance N,N-dialkylpyrrolidinium, N,N-dialkylpiperidinium or N,N-trialkylammonium agents.

Another subject of the invention is thus a composition comprising at least one molecule (I) as described above and at least one solvent. Such compositions, just like the pure compound (I), may be used in a process for extracting, detecting and assaying metals from an aqueous composition.

Another subject of the invention is a process for extracting metals in an aqueous composition, this process comprising at least one step of placing the aqueous composition in which the metal(s) is (are) present in contact with a compound of formula (I), this compound of formula (I) being able to be used for the implementation of this step either neat, when it is liquid at room temperature, or in solution. Preferably, this placing in contact comprises at least one mixing step. For example, the two compositions may be placed in the same container and stirred together using a mechanical stirrer. It is also possible to use a closed container in which the two compositions are placed, and the whole is mixed using manual or mechanical stirring means. It may also be envisaged to use a fluid-circulating device in which streams of each of the compositions are made to meet so as to allow exchange between the two phases. Devices such as those that have been described in WO2007/138180 may especially be used. In the case of microsystems, the contact between the molecule (I) and the metal ion may be performed by diffusion at the interface between the hydrophobic solvent and water.

Advantageously, the process of the invention is performed in a microfluidic device. Specifically, such devices have the advantage of being easy to transport and of using only small amounts of products (sample to be tested and chelating agent/detector). They are thus also economical and ecological. Such a device comprises at least one mixing chamber in which the sample to be tested and the chelating agent/detector of formula (I) (neat or in composition form) are placed in contact. The mixing chamber may have any configuration, provided that it allows the two compositions to be brought into contact, and it may especially be a fluid circulation channel. Such devices are described especially in Tokeshi et al., Anal. Chem. 2002, 74, 1565-1571. According to another preferred variant, the process of the invention is performed in a test tube or in a well plate.

This process may be used for scavenging and detecting any metal compound, and it especially enables the scavenging and detection of the following metals (metallic trace elements MTE): iron (Fe), lead (Pb), mercury (Hg), uranium (U), chromium (Cr), copper (Cu), silver (Ag), gold (Au), zinc (Zn), titanium (Ti), nickel (Ni), cadmium (Cd), magnesium (Mg), calcium (Ca), but also other pollutant metals such as: aluminium (Al), gallium (Ga), germanium (Ge), arsenic (As), selenium (Se), rubidium (Rb), strontium (Sr), yttrium (Y), zirconium (Zr), niobium (Nb), molybdenum (Mo), technetium (Tc), ruthenium (Ru), rhodium (Rh), palladium (Pd), indium (In), tin (Sn), antimony (Sb), tellurium (Te), caesium (Cs), barium (Ba), luthenium (Lu), hafnium (Hf), tantalum (Ta), tungsten (W), rhenium (Re), osmium (Os), iridium (Ir), platinum (Pt), thallium (Tl), plutonium (Pu).

The invention may also be performed in the context of detecting metals in a hydrophilic liquid. In this case, A⁻ will be chosen from BF₄⁻ (tetrafluoroborate) and from halogenated ions (for example Cl⁻). The groups X, Y, W, R¹, R², R³, R⁴, R⁵ and R⁶ are chosen such that the compound is hydrophilic.

Another subject of the invention is a kit for detecting metals in aqueous medium, comprising a microfluidic device and at least one compound of formula (I), whether it is in pure form or in a composition.

The placing in contact of the sample to be tested, in the form of an aqueous composition, and of the chelating agent/detector of formula (I) results in chelation of the metals present in the sample to be tested by the chelating agent, and in their extraction from the aqueous phase. This placing in contact also results in the emission of a fluorescence signal under the effect of a source of excitation, delivering a light signal whose wavelength depends on the metal, and is generally between 280 and 380 nm. The intensity of the fluorescence emission makes it possible to quantify, after calibration using a reference sample, the concentration of metals in the initial aqueous phase.

Another subject of the invention is thus a process for detecting metals in an aqueous composition, this process comprising at least one step of placing in contact the aqueous composition in which the metal(s) is (are) present in contact with a compound of formula (I), and at least one step of fluorescence measurement.

Such a process is illustrated schematically in FIG. 1: on the left, an aqueous phase containing metal ions and a solvent phase containing a molecule of formula (I) before liquid/liquid extraction. The molecule (I) fluoresces very little or not at all. On the right, after liquid/liquid extraction and complexation of metal ions by the molecule (I), the solvent phase becomes fluorescent.

Another subject of the invention is a kit for detecting metals in aqueous medium, comprising at least one compound of formula (I), whether it is in pure form or in a composition, and a fluorescence detector.

### EXAMPLES

### FIGURES

FIG. 1: schematic representation of an extraction and detection process,

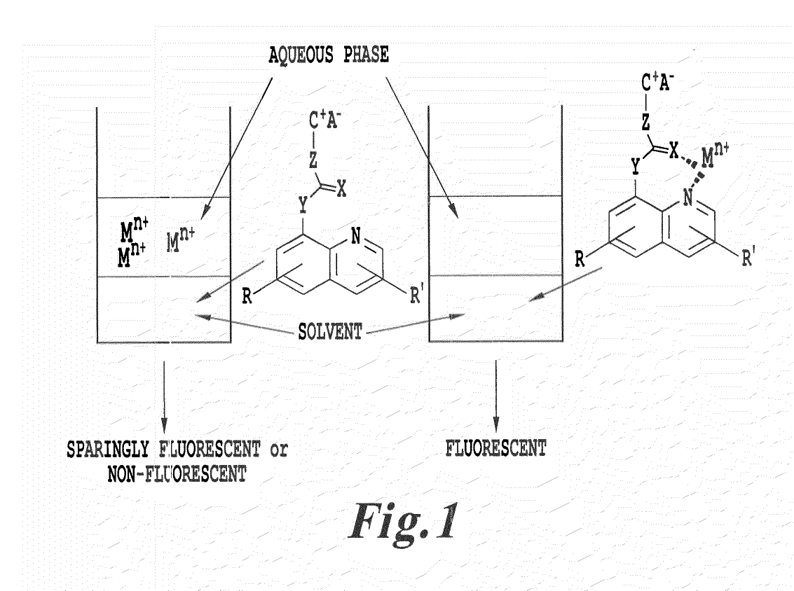

FIG. 2A: fluorescence of (5) as a function of the amount of Hg(ClO₄)₂,

FIG. 2B: ratio of the fluorescence intensities between the complex M(ClO₄)₂ with M variable and Hg(ClO₄)₂,

FIG. 3A: fluorescence of (8) in acetonitrile with addition of CuCl₂,

FIG. 3B: ratio of the fluorescence intensities between the complex MCl₂ with M variable and HgCl₂,

FIG. 4: fluorescence of (8) in [bmp] [NTf₂] before extraction a), after extraction with an aqueous HgCl₂ solution b), control with distilled water c), λ_{exc.}=380 nm,

FIG. 5: fluorescence of (5) in [bmp] [NTf₂] after extraction according to the amount of Hg(ClO₄)₂ contained in the aqueous phase, λ_{exc.}=313 nm,

FIG. 6A: schematic representation of the microfluidic circuit,

FIG. 6B: photograph of the microfluidic circuit (magnification: ⁶),

FIG. 7: fluorescence of (5) in [bmp] [NTf₂] (IL) after extraction of [Hg(ClO₄)₂] dissolved in water, in the microsystem. F.R._{water}=4 μL/minute, F.R._{IL}=0.02 μL/minute.

### MATERIALS AND METHODS

The anhydrous solvents and the reagents are obtained from Sigma-Aldrich and Roth. They are used as supplied. The ¹H and ¹³C NMR spectra were recorded on a Brüker Avance 200 spectrometer (200.13 MHz for the proton, 50 MHz for carbon).

The fluorescence emission spectra are obtained on a Perkin-Elmer LS50B spectrofluorimeter.

1-Syntheses

### Example 1

Compound (5) was synthesized in accordance with the synthetic scheme outlined in Scheme 1.

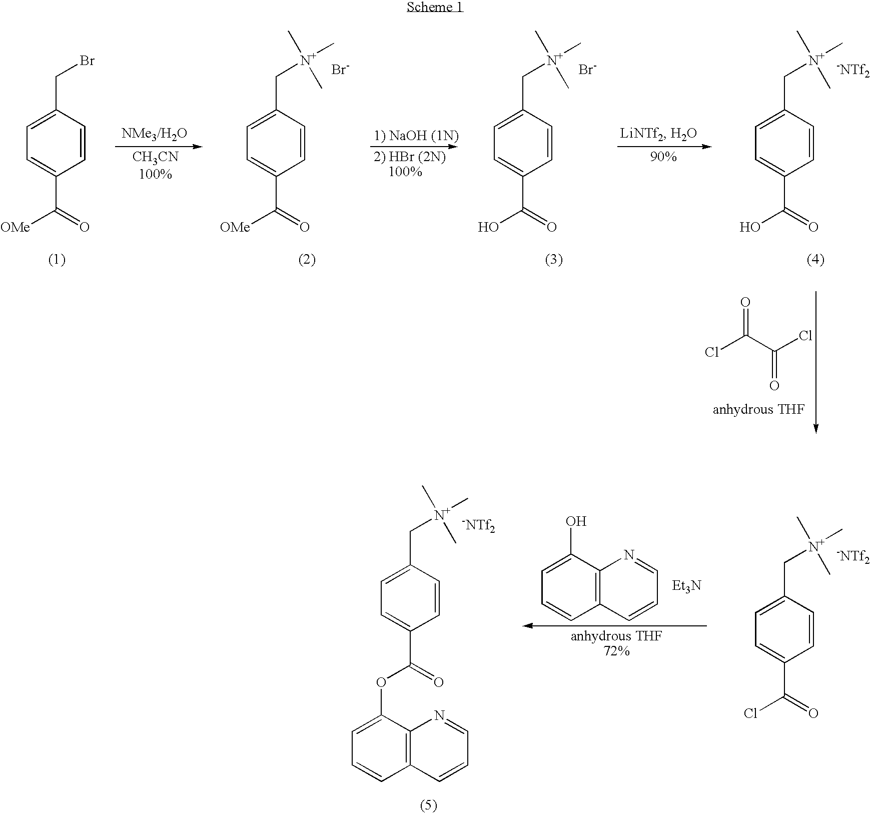

Synthesis of (2). In a Schlenck tube, a solution of trimethylamine (45% in water, 2 equivalents) is added in a single portion to a solution of methyl 4-(bromomethyl)benzoate (1) (1 equivalent) in acetonitrile (20 mL). The reaction mixture is stirred and heated at 70° C. for 15 hours. The solvent and the excess trimethylamine are then evaporated off with a vane pump. The residue is washed with ether (4×10 mL) and then dried again on the vane pump to obtain a white solid (2) (yield: 100%).

Synthesis of (3). 1 equivalent of (2) is dissolved in aqueous NaOH (1M, 1 equivalent). Deionized water is added until the dissolution of (2) is complete. The reaction mixture is stirred and heated at 80° C. for 15 hours. After cooling to room temperature, HBr (2N, 1 equivalent) is introduced. Stirring is continued for 15 minutes and the water is then evaporated off with a vane pump, while heating at 60° C., using an oil bath, for 2 hours. The residue is washed with ether (3×10 mL) and then dried under vacuum (vane pump) for 3 hours to obtain a white solid (3) (yield: 100%).

Synthesis of (4). A solution of LiNTf₂ (2 equivalents) in water is added to (3) (1 equivalent). A white precipitate is formed. The reaction mixture is stirred for 3 hours at room temperature and then filtered through a sinter funnel (porosity 4). The residue obtained is washed (3×20 mL) with water and then dried under vacuum (vane pump), while heating at 60° C. using an oil bath for 3 hours, to obtain a white solid (4) (yield: 76%).

Synthesis of (5). 6 drops of anhydrous DMF are added to a solution of (4) (1 equivalent) in anhydrous THF (10 mL). After cooling to 0° C., oxalyl chloride (1.5 equivalents) is added dropwise. Stirring is continued for 15 minutes at 0° C., and then at room temperature until the evolution of gas has ceased (3 hours). The solvent and the excess oxalyl chloride are evaporated off under vacuum (vane pump). The residue obtained is redissolved in anhydrous THF, and 8-hydroxyquinoline and then triethylamine, freshly distilled, are added. The reaction mixture is stirred for 4 hours at room temperature and then filtered through a sinter funnel (porosity 4). The filtrate is concentrated on a rotary evaporator and then dissolved in dichloromethane (20 mL). The organic phase obtained is washed (3×10 mL) with deionized water and then concentrated on a rotary evaporator and washed (3×10 mL) with ether to obtain a greenish-white solid (5) (yield: 72%, m.p.=120° C.). ¹H NMR (CD₃CN) δ 8.81 (dd, ³J=4.1 Hz, ⁴J=1.6 Hz, 1H), 8.38 (d, ³J=8.3 Hz, 2H), 8.37 (dd, ³J=8.3 Hz, ⁴J=1.6 Hz, 1H), 7.92 (dd, ³J=6.0 Hz, ³J=3.5 Hz, 1H), 7.74 (d, ³J=8.3 Hz, 2H), 7.66 (dd, ³J=6.0 Hz, 1H), 7.65 (d, ³J=3.5 Hz, 1H), 7.52 (dd, ³J=8.3 Hz, ³J=4.1 Hz, 1H), 4.5 (s, 2H), 3.06 (s, 9H); ¹³C NMR (CD₃CN) δ 165.54, 151.53, 148.30, 141.67, 137.17, 134.40, 133.83, 132.44, 131.50, 130.41, 127.38, 127.35, 123.07, 122.46, 120.81 (q, J_{CF}=320.6 Hz), 69.4, 53.5 (t, J_{CN}=3.9 Hz). MS (C⁺) 321.1.

### Example 2

The synthesis of compound (8) is performed in accordance with the process described in Scheme 2.

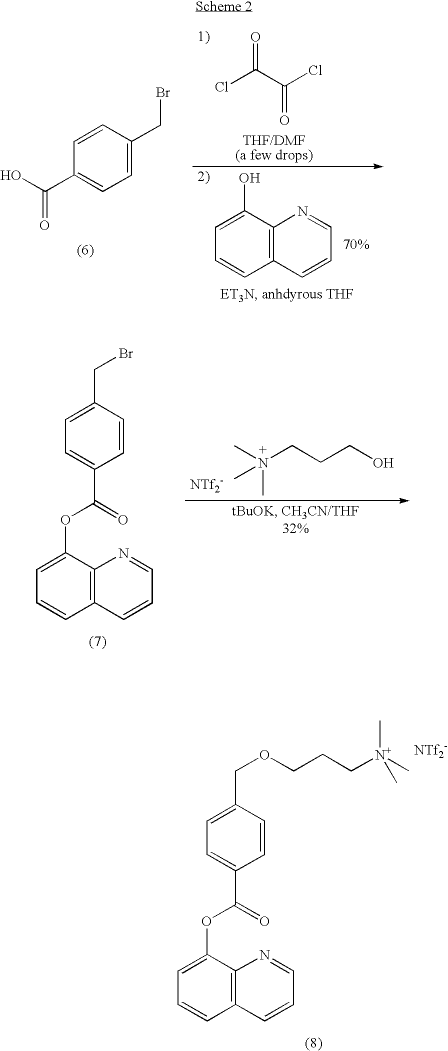

Synthesis of (7). 6 drops of anhydrous DMF are added to a solution of 4-bromomethylbenzoic acid (6) (1 equivalent) in anhydrous THF (10 mL). After cooling to 0° C., oxalyl chloride (1.5 equivalents) is added dropwise. Stirring is continued for 15 minutes at 0° C. and then at room temperature until the evolution of gas has ceased (3 hours). The solvent and the excess oxalyl chloride are evaporated off on a vane pump. The residue obtained is redissolved in anhydrous THF, and 8-hydroxyquinoline and then triethylamine, freshly distilled, are added. The reaction mixture is stirred for 4 hours at room temperature and then filtered through a sinter funnel (porosity 4). The filtrate is concentrated on a rotary evaporator. (7) is isolated by purification on a column of silica (eluent: 9/1 CH₂Cl₂/EtOAc) to give a nacreous white solid (yield: 70%). m.p.=137° C.

Synthesis of (8). Under argon, the base KOtBu (1.1 equivalents) is added to a solution of (3-hydroxypropyl)trimethylammonium bis(trifluoromethane-sulfonamide) in anhydrous acetonitrile. After stirring for 1 hour 30 minutes at room temperature, a solution of (7) in a 1/1 THF/CH₃CN mixture is added to the reaction medium. Stirring is continued for 24 hours at room temperature, and the reaction mixture is then filtered through a filter paper. The filtrate is concentrated and then washed five times (5×10 mL) with ether. (9) is isolated by purification on a column of C₁₈ silica (eluent: 3/3/4 CH₃CN/CH₃OH/H₂O) to give a viscous brown oil (yield: 32%). ¹H NMR (acetone-d₆) δ 8.96 (dd, ³J=4.1 Hz, ⁴J=1.6 Hz, 1H), 8.34 (dd, ³J=8.3 Hz, ⁴J=1.6 Hz, 1H), 8.11 (d, ³J=8.3 Hz, 2H), 7.80 (d, ³J=8.3 Hz, 2H), 7.60 (d, ³J=4.1 Hz, 1H), 7.57-7.49 (m, 2H), 7.31 (dd, ³J=6.7 Hz, ⁴J=2.2 Hz, 1H), 5.52 (s, 2H), 4.53 (t, ³J=5.7 Hz, 2H), 3.94-3.83 (m, 2H), 3.47 (s, 9H), 2.6-2.46 (m, 2H). ¹³C NMR (acetone-d₆) δ 167.09, 156.10, 150.71, 144.95, 142.34, 137.29, 131.22, 131.163, 130.84, 128.26, 128.26, 123.43, 121.96, 121.7 (q, J_{CF}=321.4 Hz), 112.04, 71.24, 65.69 (t, J_{CN}=3.5 Hz), 63.05, 54.51 (t, J_{CN}=3.9 Hz), 24.32. MS (C⁺) 379.3.

### Example 3

The synthesis of compound (9) is performed in accordance with the process described in Scheme 3:

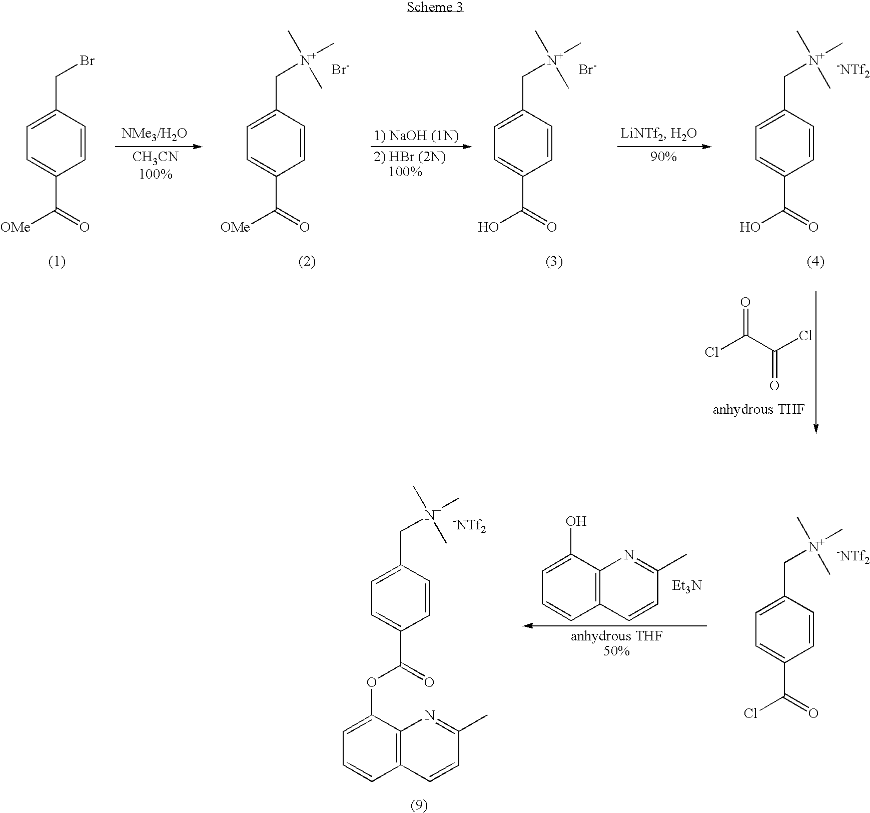

Synthesis of (9). 6 drops of anhydrous DMF are added to a solution of (4) (1 equivalent, synthesis described previously) in anhydrous THF (10 mL). After cooling to 0° C., oxalyl chloride (1.5 equivalents) is added dropwise. Stirring is maintained for 15 minutes at 0° C. and then at room temperature until the evolution of gas has ceased (3 hours). The solvent and the excess oxalyl chloride are evaporated off under vacuum (vane pump). The residue obtained is redissolved in anhydrous THF, and 8-hydroxyquinaldine and then triethylamine, freshly distilled, are added. The reaction mixture is stirred for 4 hours at room temperature and then filtered through a sinter funnel (porosity 4). The filtrate is concentrated on a rotary evaporator, washed (3×10 mL) with ether and then dissolved in dichloromethane (20 mL). The organic phase obtained is washed (3×10 mL, with deionized water and then concentrated on a rotary evaporator to obtain a greenish-white solid (9) (yield: 50%, m.p.: 120° C.). ¹H NMR (acetone-d₆) δ 8.45 (d, ³J=8.3 Hz, 2H), 8.35 (d, ³J=8.6 Hz, 1H), 8.00 (d, ³J=8.3 Hz, 2H), 7.94 (dd, ³J=5.4 Hz, ³J=4.1 Hz, 1H), 7.66 (d, ³J=5.4 Hz, 1H), 7.51 (d, ³J=8.6 Hz, 1H), 5.01 (s, 2H), 3.52 (s, 9H), 2.63 (s, 3H). ¹³C NMR (acetone-d₆): δ 165.85, 160.9, 148.85, 137.8, 135.1, 134.8, 133.6, 132.25, 129.6, 127.6, 127.0, 124.4, 123.1 (q, J_{CF}=320.6 Hz), 70.2, 54.2 (t, J_{CN}=3.9 Hz), 26.4.

### Example 4

The synthesis of compound (14) is performed in accordance with the process described in Scheme 4:

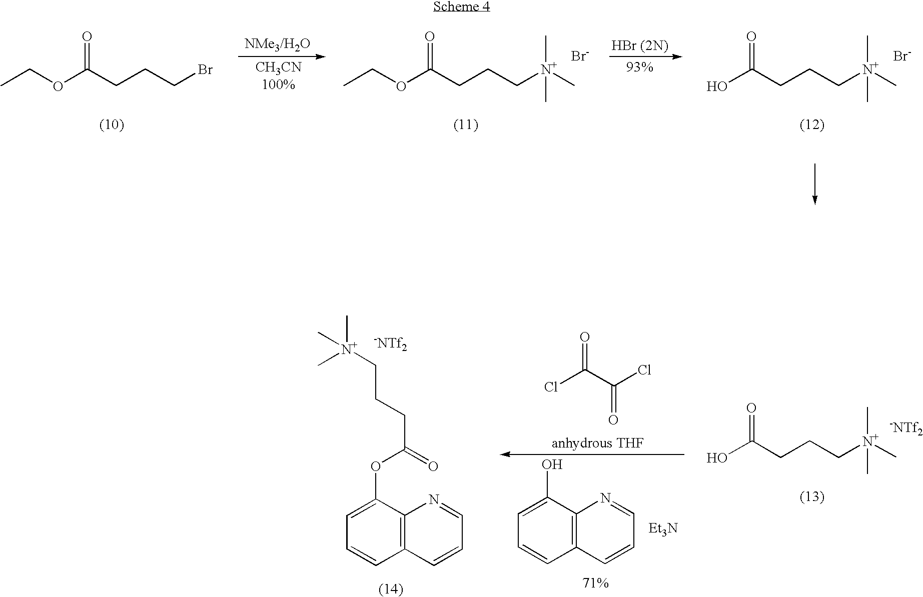

Synthesis of (11). In a Schlenck tube, a trimethylamine solution (45% in water, 2 equivalents) is added in a single portion to a solution of ethyl 4-bromobutyrate (1 equivalent) in acetonitrile (20 mL). The reaction mixture is stirred and heated at 80° C. for 15 hours. The solvent and the excess trimethylamine are then evaporated off with a vane pump. The residue is washed with ether (4×10 mL) and then dried again with the vane pump to obtain a white solid (12) (yield: 100%).

Synthesis of (12). 1 equivalent of (11) is dissolved in aqueous HBr (2N, 1 equivalent). Deionized water is added until the dissolution of (11) is complete. The reaction mixture is stirred and heated at 80° C. for 15 hours, and the water is then evaporated off with a vane pump, while heating at 60° C. using an oil bath, for 2 hours. The residue is washed with ether (3×15 mL) and then once with acetone (15 mL) and dried under vacuum (vane pump) to give a white solid (12) (yield: 93%, m.p.=196° C.)

Synthesis of (13). A solution of LiNTf₂ (1.5 equivalents) in water is added to (12) (1 equivalent). The reaction mixture is stirred for 3 hours at room temperature. Two phases are obtained and transferred into a separating funnel. The lower phase (ionic liquid) is separated out and the aqueous phase is then washed twice (2×10 mL) with CH₂Cl₂. After evaporating off the solvent, a pale yellow oil is obtained (13) (yield: 71%).

Synthesis of (14). 6 drops of anhydrous DMF are added to a solution of (13) (1 equivalent) in anhydrous THF (10 mL). After cooling to 0° C., oxalyl chloride (1.5 equivalents) is added dropwise. Stirring is continued for 15 minutes at 0° C. and then at room temperature until the evolution of gas has ceased (2 hours). The solvent and the excess oxalyl chloride are evaporated off under vacuum (vane pump). The residue obtained is redissolved in anhydrous THF, and 8-hydroxyquinoline and then triethylamine, freshly distilled, are added. The reaction mixture is stirred for 4 hours at room temperature and then filtered through a sinter funnel (porosity 4). The filtrate is concentrated on a rotary evaporator, washed (3×10 mL) with ether and then dissolved in dichloromethane (20 mL). The organic phase obtained is washed three times (3×10 mL) with deionized water and then concentrated on a rotary evaporator to give a very viscous brown-green oil (14) (yield: 78%). ¹H NMR (acetone-d₆) δ 8.98 (dd, ³J=4.1 Hz, ⁴J=1.6 Hz, 1H), 8.47 (dd, ³J=8.6 Hz, ⁴J=1.6 Hz, 1H), 7.96 (dd, ³J=8.3 Hz, ⁴J=1.6 Hz, 1H), 7.73-7.55 (m, 3H), 3.97-3.88 (m, 2H), 3.5 (s, 9H), 3 (t, ³J=, 2H), 2.42-2.57 (m, 2H), ¹³C NMR (acetone-d₆): δ 171.98, 152.08, 149.12, 142.51, 137.90, 131.27, 127.97, 127.74, 123.68, 123.04, 121.73 (J_{CF}=321.6 Hz), 67.24 (t, J_{CN}=3.1 Hz), 54.53 (t, J_{CN}=4.1 Hz), 20.23.

### Example 5

The synthesis of compound (15) is performed in accordance with the process described in Scheme 5:

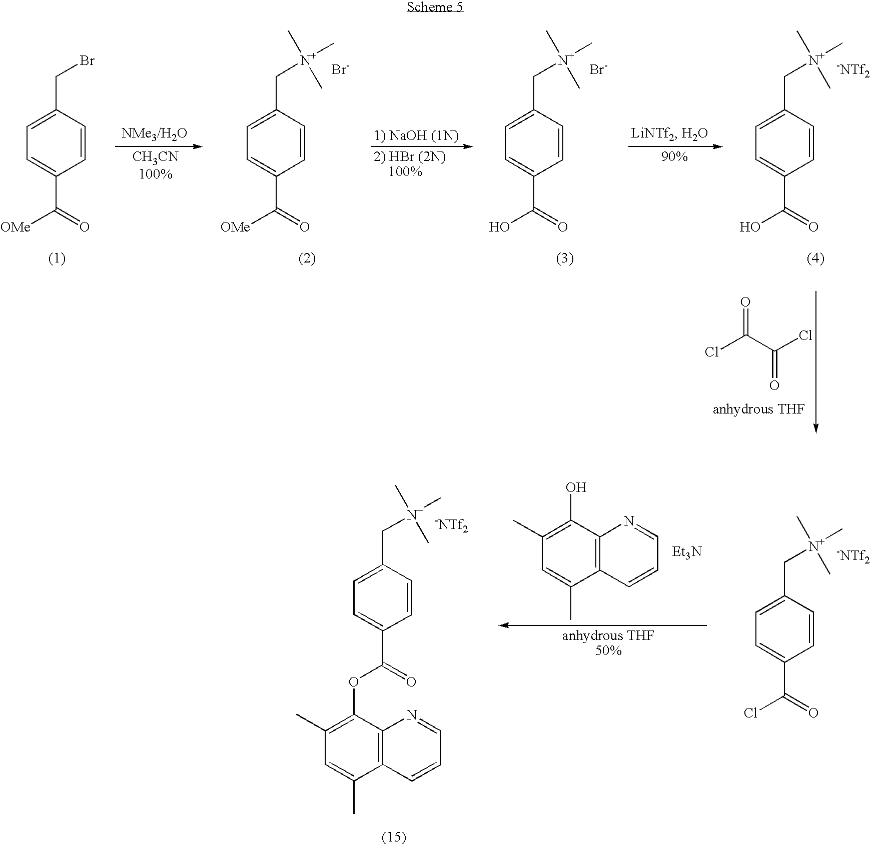

Synthesis of (15). 6 drops of anhydrous DMF are added to a solution of (4) (1 equivalent, synthesis described previously in Example 1) in anhydrous THF (10 mL). After cooling to 0° C., oxalyl chloride (1.5 equivalents) is added dropwise. Stirring is continued for 15 minutes at 0° C. and then at room temperature until the evolution of gas has ceased (3 hours). The solvent and the excess oxalyl chloride are evaporated off under vacuum (vane pump). The residue obtained is redissolved in anhydrous THF, and 5,7-dimethyl-8-hydroxyquinoline and then triethylamine, freshly distilled, are added. The reaction mixture is stirred for 4 hours at room temperature and then filtered through a sinter funnel (porosity 4). The filtrate is concentrated on a rotary evaporator, washed (3×10 mL) with ether and then dissolved in dichloromethane (20 mL). The organic phase obtained is washed (3×10 mL) with deionized water and then concentrated on a rotary evaporator to give a very viscous greenish oil (15) (yield: 63%). ¹H NMR (CD₃CN): δ 8.74 (d, ³J=3.5 Hz, 1H), 8.36 (d, ³J=7.6 Hz, 2H), 8.28 (d, ³J=8.3 Hz, 1H), 7.68 (d, ³J=7.6 Hz, 2H), 7.45 (dd, ³J=8.3 Hz, ³J=3.5 Hz, 1H), 7.35 (s, 1H), 4.46 (s, 2H), 3.04 (s, 9H), 2.64 (s, 3H), 2.38 (s, 3H). ¹³C NMR (CD₃CN): δ 165.2, 150.8, 141.8, 134.4, 134.2, 133.9, 133.8, 132.5, 131.5, 131.3, 130.5, 127.8, 124.0, 121.7, 120.8 (q, J_{CF}=319.8 Hz), 69.4, 53.6 (t, J_{CN}=3.9 Hz), 18.4, 16.3.

### Example 6

The synthesis of compound (17) is performed in accordance with the process described in Scheme 6:

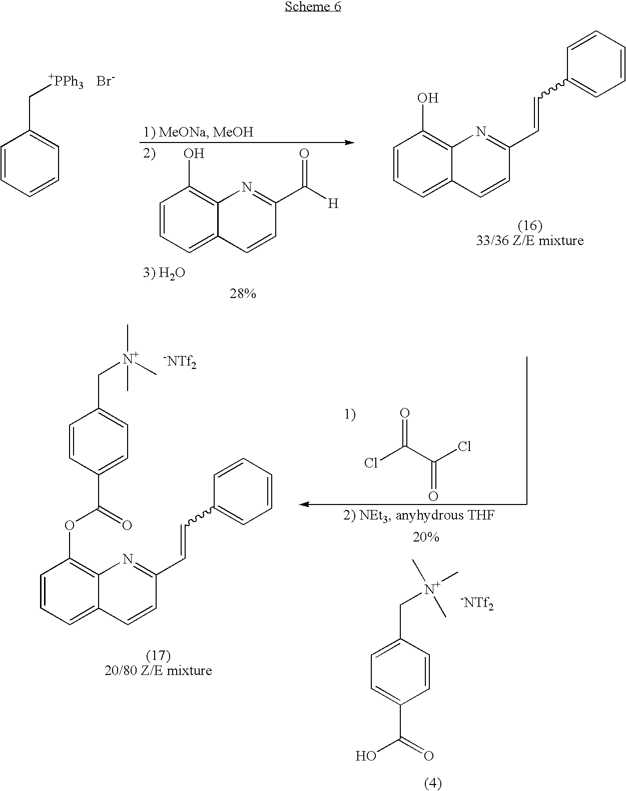

Synthesis of (16), 2-styryl-8-hydroxyquinoline. Sodium methoxide (0.5 M in methanol, 1.1 equivalents) is added slowly to a solution of benzyltriphenylphosphonium bromide (1.1 equivalents) in methanol (5 mL). After stirring for 20 minutes at room temperature, 2-carbaldehyde-8-hydroxyquinoline is added. The mixture is maintained at the reflux point of methanol for 6 hours. After cooling to room temperature and hydrolysing by adding saturated NaCl (25 mL), the medium is neutralized with dilute HCl. The products are extracted with dichloromethane (3×30 mL). The resulting organic phase is washed with water (2×20 mL), dried over MgSO₄ and then concentrated on a rotary evaporator. Purification on silica gel (eluent: dichloromethane) gives compound (16) (33/66 Z/E mixture) in the form of a viscous yellow oil. (yield: 28%) ¹H NMR (CD₃CN) for the Z isomer: δ 8.25 (brs, 1H), 8.15 (d, ³J=8.6 Hz, 1H), 7.49-7.34 (m, 8H), 7.1 (dd, ³J=6.7 Hz, ²J=2.25 Hz, 1H), 7.09 (d, ¹J=12.4 Hz, 1H), 6.89 (d, ¹J=12.4 Hz). For the E isomer: 8.90 (brs, 1H), 8.33 (d, ³J=8.6 Hz, 1H), 8.14 (d, ¹J=16.2 Hz, 1H), 7.84 (d, ³J=8.6 Hz, 1H), 7-79-7.75 (m, 2H), 7.55 (d, ¹J=16.2 Hz, 1H), 7.53-7.33 (m, 5H), 7.16 (dd, ³J=6.7 Hz, ²J=2.25 Hz, 1H).

Synthesis of (17). 6 drops of anhydrous DMF are added to a solution of (4) (1 equivalents, synthesis described previously in Example 1) in anhydrous THF (10 mL). After cooling to 0° C., oxalyl chloride (1.5 equivalents) is added dropwise. Stirring is continued for 15 minutes at 0° C., and then at room temperature until the evolution of gas has ceased (3 hours). The solvent and the excess oxalyl chloride are evaporated off under vacuum (vane pump). The residue obtained is redissolved in anhydrous THF, and 2-styry1-8-hydroxyquinoline (16) (33/66 Z/E mixture) and then triethylamine, freshly distilled, are added. The reaction mixture is stirred for 4 hours at room temperature and then filtered through a sinter funnel (porosity 4). The filtrate is concentrated on a rotary evaporator, washed (3×10 mL) with ether and then dissolved in dichloromethane (20 mL). The organic phase obtained is washed (3×10 mL) with deionized water and then concentrated on a rotary evaporator to give a viscous greenish oil (17) (20/80 Z/E mixture, yield: 20%, m.p.:). ¹H NMR (CD₃CN) for the mixture: δ 8.45 (d, ³J=8.3 Hz, 2H_{E}), 8.32 (d, ³J=8.6 Hz, 1H_{E}), 8.26 (d, ³J=8.3 Hz, 2H_{z}), 8.13 (d, ³J=8.6 Hz, 1H_{z}), 7.87-7.18 (m, 13H_{E}+11H_{z}), 6.87 (d, ¹J=12.7 Hz, 1 Hz), 6.67 (d, ¹J=12.7 Hz, 1 Hz), 4.54 (s, 2H_{E}), 4.49 (s, 2H_{z}), 3.08 (s, 9H_{E}), 3.06 (s, 9H_{z}).

2. Spectrometric Measurements

The object of these measurements is to show that the molecule of general formula (I) allows simultaneous chelation and direct detection by fluorescence emission independently of the extraction.

### Example of Selective Detection

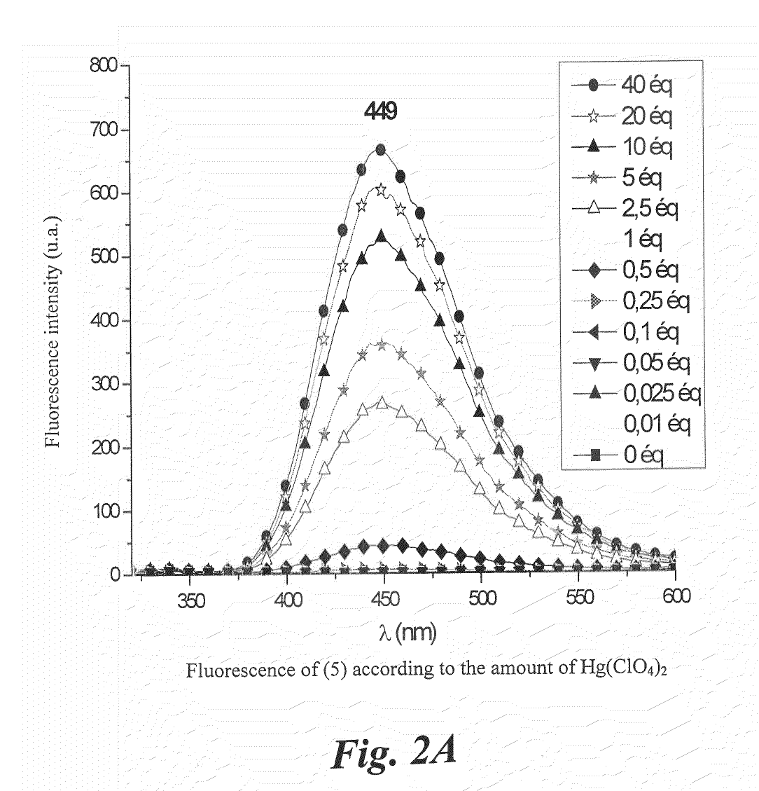

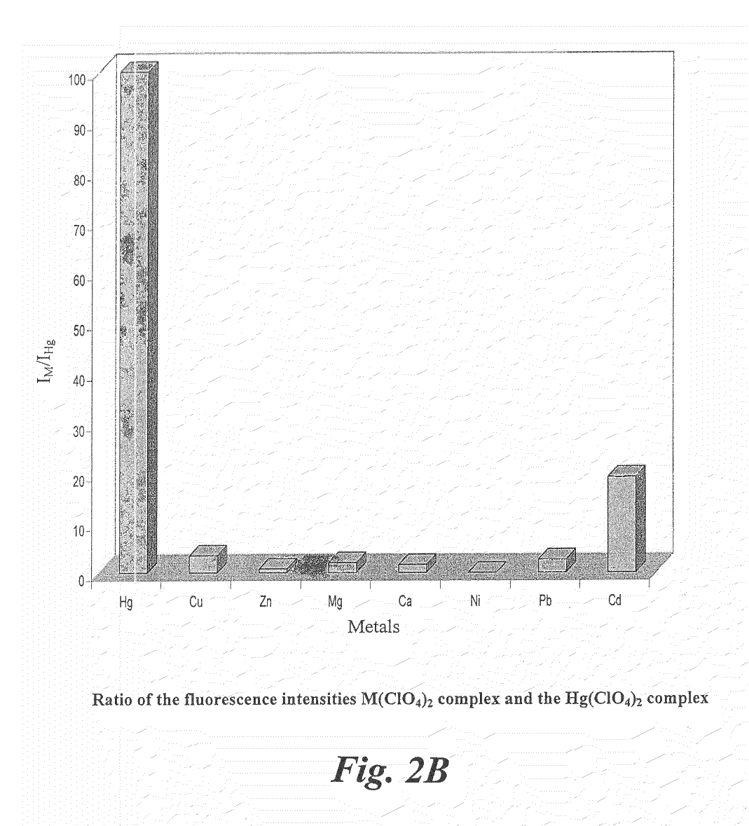

A solution of (5) in acetonitrile (c=10⁻⁵M) is titrated by increasing the amount of metal salts dissolved in water. After each addition of metal, the fluorescence measurements are taken between 315 and 600 nm (λ_{exc}=310 nm). The number of equivalents of metal salt ranges between 0 and 40 equivalents of ligand (5). This means that the [metal salt]/[ligand (5)] concentration ratio ranges between 0 and 40. As shown in FIG. 2, the fluorescence signal at 449 nm increases as the number of equivalents of Hg(ClO₄)₂ increases. Selectivity tests with other metals showed that the order of selectivity is as follows: Hg²⁺>>Cd²⁺>Cu²⁺>Pb²⁺>Mg²⁺>Ca²⁺>Zn²⁺>Ni²⁺.

### Example of Non-Selective Detection

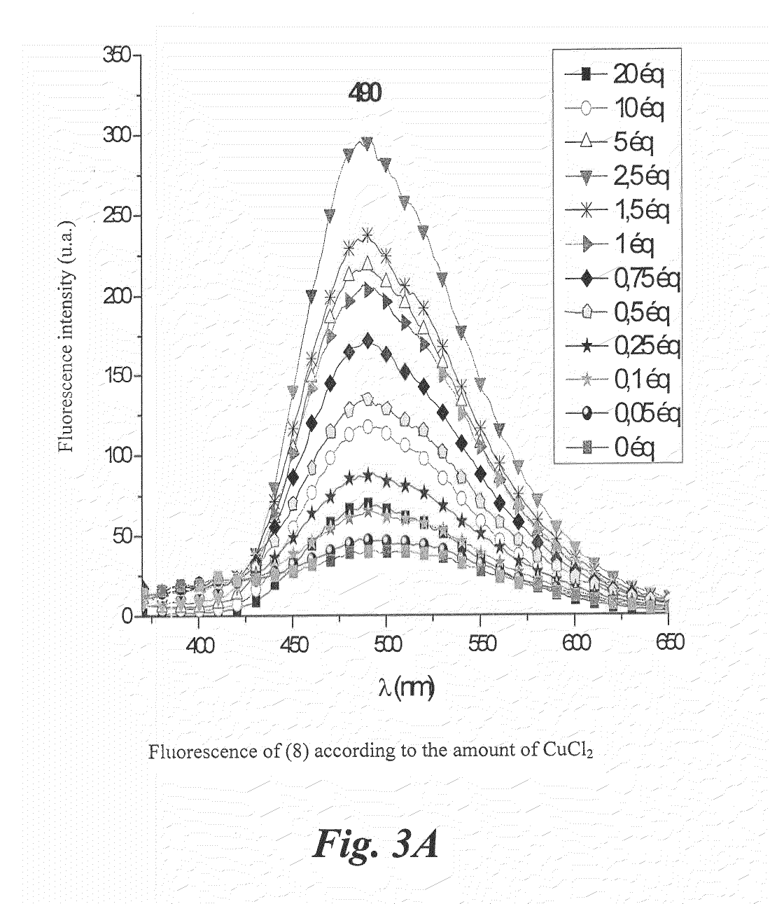

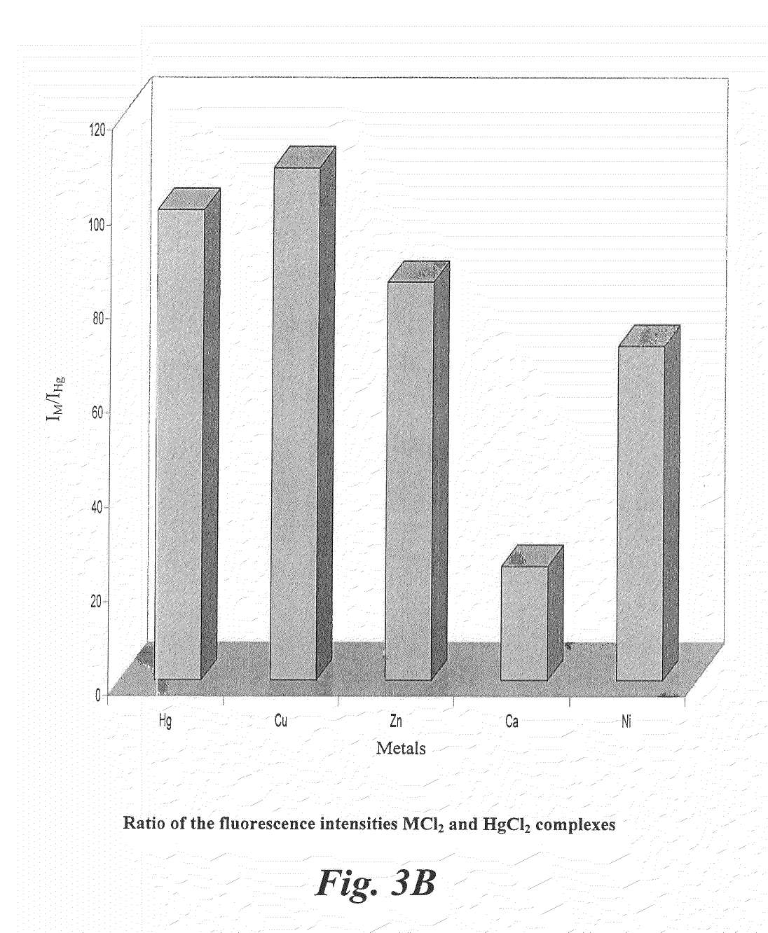

A solution of (8) in acetonitrile (c=5.10⁻⁴M) is titrated by increasing the amount of metal salts (CuCl₂) dissolved in water. After each addition of metal, the fluorescence measurements are taken between 370 and 650 nm (λ_{exc}=365 nm). The number of equivalents of metal salt ranges between 0 and 40 equivalents of ligand (8), i.e. the [metal salt]/[ligand (5)] concentration ratio ranges between 0 and 40. As shown in FIG. 3, the fluorescence signal at 490 nm increases as the number of equivalents of CuCl₂ increases. Selectivity tests with other metals showed that this molecule (8) was not selective for any metal in particular.

### Example of Aqueous-Phase Extraction—Ionic Liquid Containing a Task-Specific Ammonium Salt (I)

### Example E1

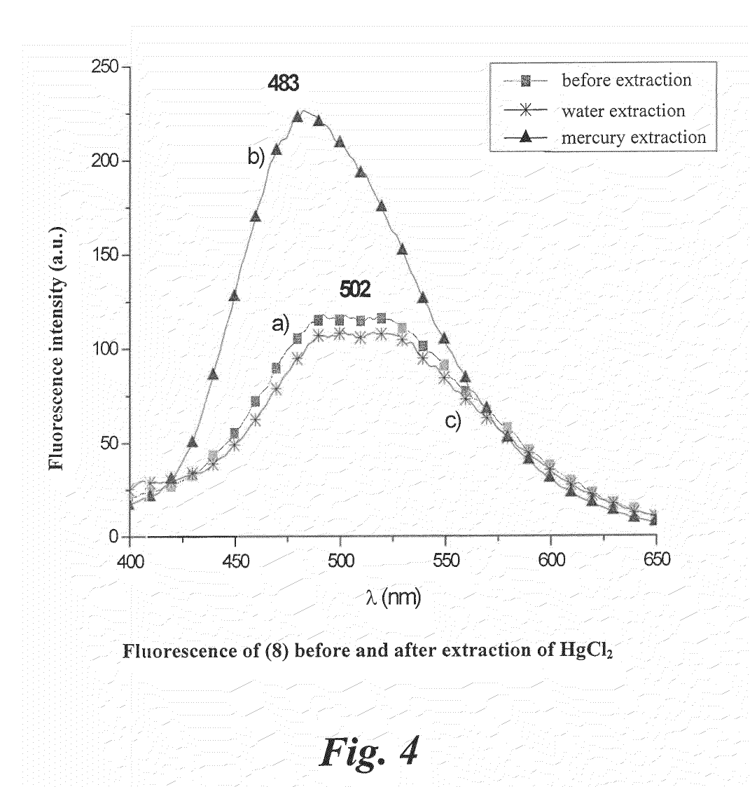

1 ml of an aqueous HgCl₂ solution of known concentration (10⁻²M) is added to 1 ml of a solution of (8) (5.0×10⁻⁴M in an ionic liquid (butylmethylpyrroli-dinium bis (trifluoromethanesulfonamide) [bmp] [NTf₂])). The two-phase system is stirred for five minutes using a vortex mixer until equilibrium is reached. After extraction, the two phases are allowed to separate by settling and the fluorescence of the phase constituted of ionic liquid is then measured between 370 nm and 650 nm. A fluorescence signal is emitted at 483 nm, as shown in FIG. 4. In this figure, the fluorescence of (8) in [bmp] [NTf₂] is observed before extraction a), after extraction with aqueous HgCl₂ solution b), control with distilled water c), λ_{exc}=380 nm.

### Example E2

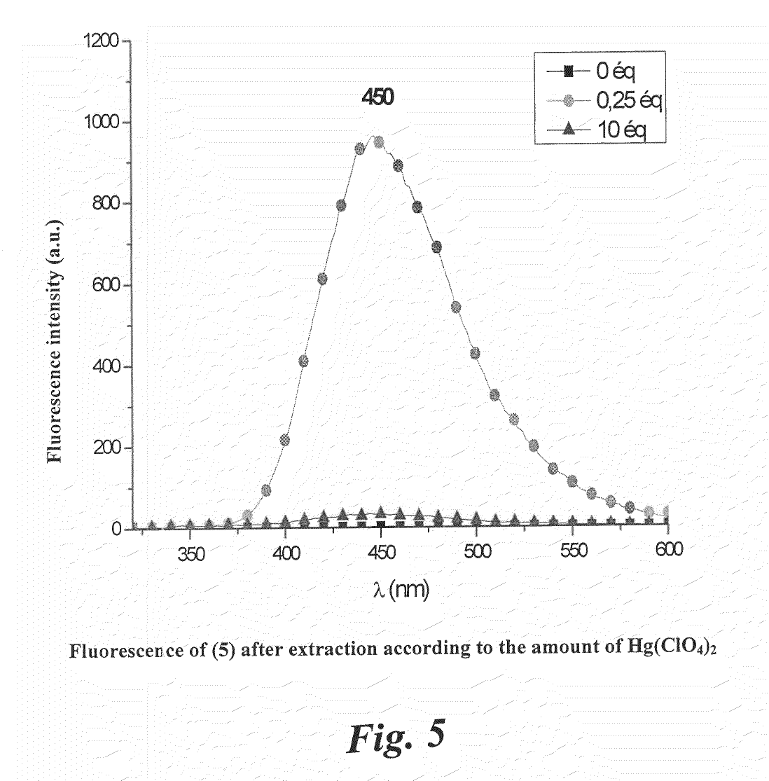

1 ml of an aqueous Hg(ClO₄)₂ solution of known concentration is added to 1 ml of a solution of (5) (1.0×10⁻⁴M) in an ionic liquid (butylmethylpyrroli-dinium bis (trifluoromethanesulfonamide) [bmp] [NTf₂]). The two-phase system is stirred for 2 minutes using a vortex mixer in order to reach equilibrium. After extraction, the two phases are separated by centrifugation (2 minutes, 2000 rpm) and the fluorescence of the phase constituted of ionic liquid is then measured between 320 nm and 600 nm. A fluorescence signal is emitted at 450 nm, as shown in FIG. 5. In this figure, the fluorescence of (5) in [bmp] [NTf₂] is observed after extraction according to the amount of Hg(ClO₄)₂ contained in the aqueous phase, λ_{exc}=313 nm.

### Example E3

Liquid/liquid extraction in a microsystem and detection by fluorescence: example of part of a “microfluidic kit”.

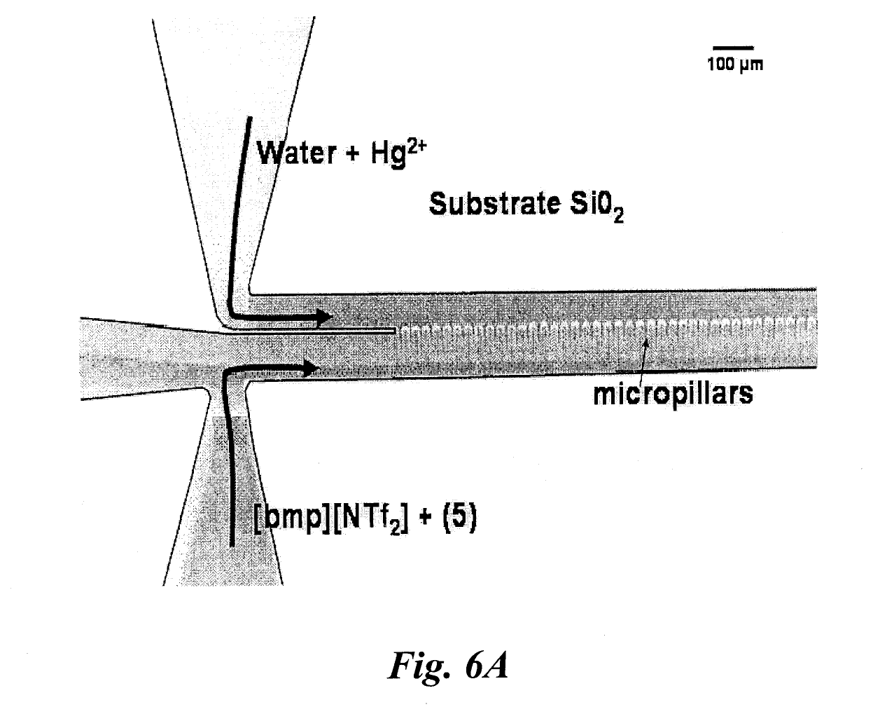

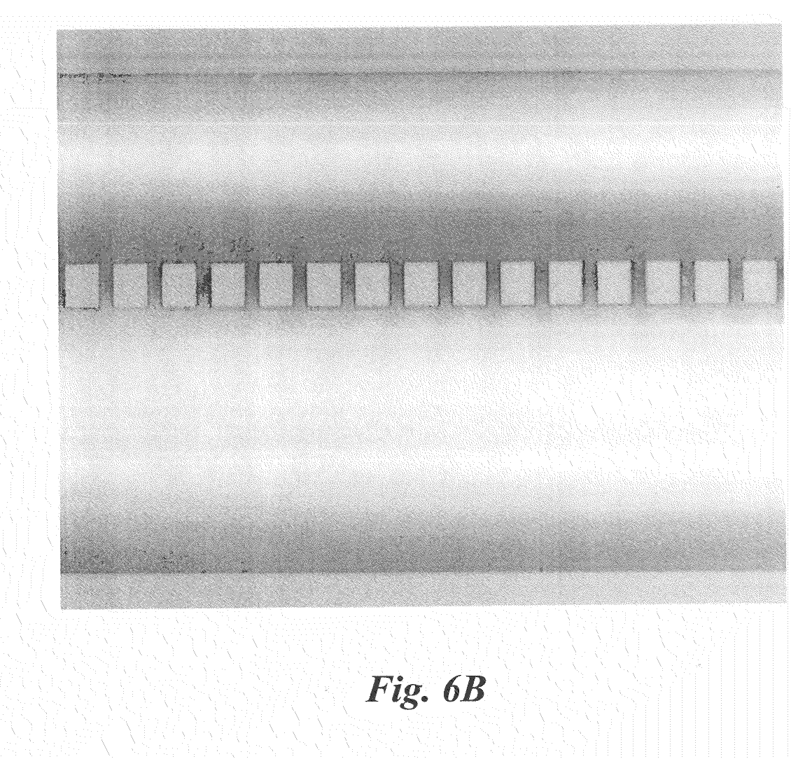

In the device shown in FIGS. 6A and 6B, the chip consists of a multi-channel microfluidic system etched in silicone and of a Pyrex lid for creating a cavity. The two-phase interface is stabilized with vertical square micropillars and the exchanges of molecular species are ensured by a series of interfaces. The chip is composed of two channels 5 mm long, 150 μm deep and 108 and 150 μm wide, respectively, for water and for the ionic liquid. The micropillars are of 28×28 μm and are spaced 9.4 μm apart.

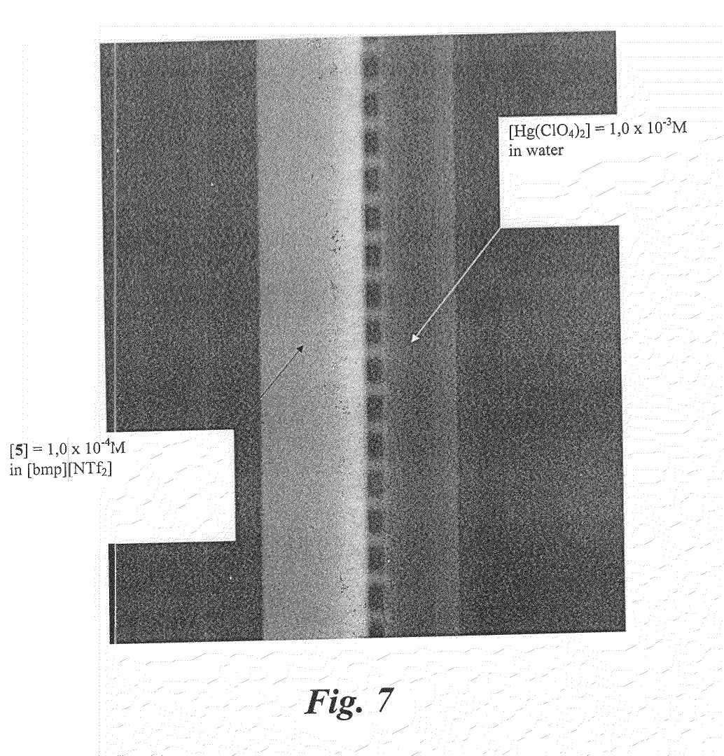

Extraction protocol: the aqueous solution containing mercury ions Hg(ClO₄)₂ was introduced at a chosen flow rate (of between 4 and 5 μL/minute) into the channel 108 μm wide. Next, the solution of (5) in [bmp] [NTf₂] was introduced at a chosen flow rate (0.02 μL/minute) into the channel 150 μm wide. After filling, optical detection was commenced using an epifluorescence microscope. The two streams ensured the stability of the interface and were maintained throughout the experiment. The fluorescence images (FIG. 7) are taken every 10 minutes so as to monitor the evolution of the fluorescence in the liquid ionic phase.

1. Molecule of formula (I): 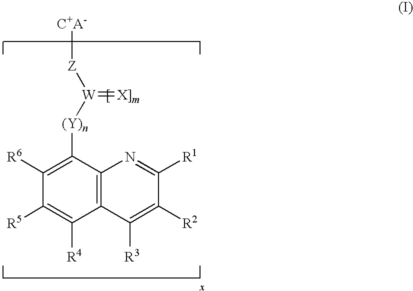
in which:
X represents a group chosen from: O, NR, S, PR, Se; and R represents a group chosen from: H, a C₁-C₁₂ alkyl group optionally comprising one or more heteroatoms, a C₂-C₁₂ alkenyl group optionally comprising one or more heteroatoms, a C₆-C₁₂ aryl group optionally comprising one or more heteroatoms, a C₆-C₁₂ aralkyl group optionally comprising one or more heteroatoms;
Y represents a group chosen from: O, S, NR′, PR′, CR′R″, a C₁-C₁₂ alkanediyl group optionally comprising one or more heteroatoms, a C₂-C₁₂ alkenediyl group optionally comprising one or more heteroatoms, a C₆-C₁₂ aryl group optionally comprising one or more heteroatoms, a C₆-C₁₂ aralkanediyl group optionally comprising one or more heteroatoms; and R′ and R″, which may be identical or different, represent a group chosen from: H, a C₁-C₁₂ alkyl group optionally comprising one or more heteroatoms, a C₂-C₁₂ alkenyl group optionally comprising one or more heteroatoms, a C₆-C₁₂ aralkyl group optionally comprising one or more heteroatoms, a C₆-C₁₂ aryl group optionally comprising one or more heteroatoms;
n is an integer chosen from: 0, 1;
n is an integer chosen from: 1, 2;
x is an integer chosen from: 1 and 2;
W represents an atom chosen from: C and S;
Z represents a group chosen from: a C₁-C₁₂ alkanediyl group optionally comprising one or more heteroelements, a C₂-C₁₂ alkenediyl group optionally comprising one or more heteroelements, a C₆-C₂₀ aralkanediyl group optionally comprising one or more heteroelements, a C₆-C₁₂ aryl group optionally comprising one or more heteroelements, the heteroelement(s) being chosen from: Cl, Br, O, S, NR″′, PR″′; and R″′ represents a group chosen from: H, a C₁-C₁₂ alkyl group, a C₂-C₁₂ alkenyl group, a C₆-C₁₂ aryl group and a C₆-C₁₂ aralkyl group;
R¹, R², R³, R⁴, R⁵ and R⁶, which may be identical or different, each represent a group chosen from the following list: H, Cl, Br, a C₁-C₁₂ alkyl group optionally comprising one or more heteroelements, a C₂-C₁₂ alkenyl group optionally comprising one or more heteroelements, a C₆-C₁₂ aryl group optionally comprising one or more heteroelements, a C₆-C₁₂ aralkyl group optionally comprising one or more heteroelements, OH, a group OR⁷, NH₂, a group NR⁷R⁸, SH, a group SR⁷; and R⁷ and R⁸ are chosen, independently of each other, from the following list: a C₁-C₁₂ alkyl group, a C₂-C₁₂ alkenyl group, a C₆-C₁₂ aryl group, a C₆-C₁₂ aralkyl group; the heteroelement(s) being chosen from: Cl, Br, O, S, NR″′, PR″′; and R″′ represents a group chosen from: H, a C₁-C₁₂ alkyl group, a C₂-C₁₂ alkenyl group, a C₆-C₁₂ aryl group and a C₆-C₁₂ aralkyl group,
→C⁺A⁻ represents a task-specific onium salt,
when x=1, C⁺A⁻ may be bonded to Z either via the anion A⁻ or via the cation C⁺,
when x=2, the anion A⁻ and the cation C⁺ are each bonded to a group Z, the two groups 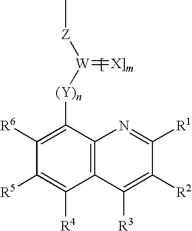
being identical or different,
and A⁻ represents a group chosen from: hexafluorophosphate (PF₆⁻), bis(trifluoromethanesulfon-amide) (NTf₂⁻), tris(pentafluoroethyl)trifluorophosphate (FAP), triflate (TfO⁻¹) and nonaflate (NfO⁻).

2. Molecule according to claim 1, in which C⁺A⁻ represents a compound chosen from ammonium, phosphonium and sulfonium salts and also salts resulting from the quaternization of an amine, a phosphine, a thioether or a heterocycle containing one or more heteroatoms such as S, N or P.

3. Molecule according to claim 1 or claim 2, in which C⁺ is chosen from ammonium, pyrrolidinium, phosphonium, imidazolium, pyridinium, piperidinium and guanidinium ions.

4. Molecule according to any one of claims 1 to 3, in which one or more of the following conditions are satisfied:
represents a group chosen from: O, S,
Y represents a group chosen from: O, S, NH, a C₁-C₆ alkanediyl group,
n=1,
m=1,
x=1,
W represents a carbon atom: C,
Z represents a group chosen from: a C₁-C₆ alkanediyl group optionally comprising one or more heteroelements, a C₆-C₂₀ aralkanediyl group optionally comprising one or more heteroelements, the heteroelement(s) being chosen from: O, S, NH,
R¹, R², R³, R⁴, R⁵ and R⁶, which may be identical or different, each represent a group chosen from the following list: H, a C₁-C₆ alkyl group,
C⁺ represents a group

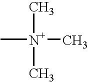

5. Molecule according to any one of claims 1 to 4, which belongs to the following list:

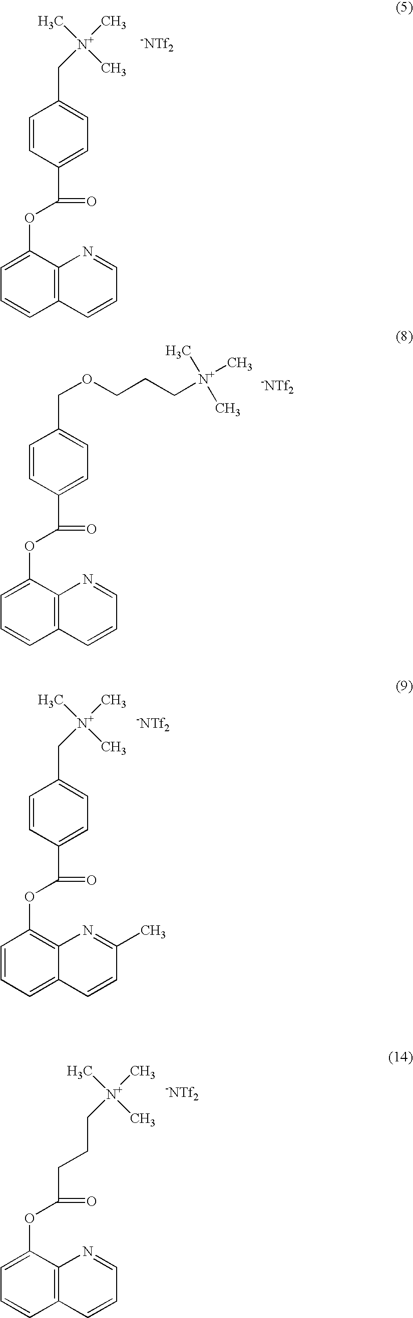

6. Composition comprising at least one molecule of formula (I): 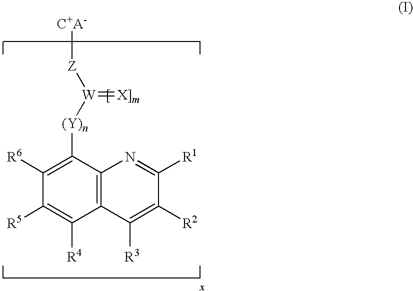
in which
X represents a group chosen from: O, NR, S, PR, Se; and R represents a group chosen from: H, a C₁-C₁₂ alkyl group optionally comprising one or more heteroatoms, a C₂-C₁₂ alkenyl group optionally comprising one or more heteroatoms, a C₆-C₁₂ aryl group optionally comprising one or more heteroatoms, a C₆-C₁₂ aralkyl group optionally comprising one or more heteroatoms;
Y represents a group chosen from: O, S, NR′, PR′, CR′R″, a C₁-C₁₂ alkanediyl group optionally comprising one or more heteroatoms, a C₂-C₁₂ alkenediyl group optionally comprising one or more heteroatoms, a C₆-C₁₂ aryl group optionally comprising one or more heteroatoms, a C₆-C₁₂ aralkanediyl group optionally comprising one or more heteroatoms; and R′ and R″, which may be identical or different, represent a group chosen from: H, a C₁-C₁₂ alkyl group optionally comprising one or more heteroatoms, a C₂-C₁₂ alkenyl group optionally comprising one or more heteroatoms, a C₆-C₁₂ aralkyl group optionally comprising one or more heteroatoms, a C₆-C₁₂ aryl group optionally comprising one or more heteroatoms;
n is an integer chosen from: 0, 1;
m is an integer chosen from: 1, 2;
x is an integer chosen from: 1 and 2;
W represents an atom chosen from: C and S;
Z represents a group chosen from: a C₁-C₁₂ alkanediyl group optionally comprising one or more heteroelements, a C₂-C₁₂ alkenediyl group optionally comprising one or more heteroelements, a C₆-C₂₀ aralkanediyl group optionally comprising one or more heteroelements, a C₆-C₁₂ aryl group optionally comprising one or more heteroelements, the heteroelement(s) being chosen from: Cl, Br, O, S, NR″′, PR″′; and R″′ represents a group chosen from: H, a C₁-C₁₂ alkyl group, a C₂-C₁₂ alkenyl group, a C₆-C₁₂ aryl group and a C₆-C₁₂ aralkyl group;
R¹, R², R³, R⁴, R⁵ and R⁶, which may be identical or different, each represent a group chosen from the following list: H, Cl, Br, a C₁-C₁₂ alkyl group optionally comprising one or more heteroelements, a C₂-C₁₂ alkenyl group optionally comprising one or more heteroelements, a C₆-C₁₂ aryl group optionally comprising one or more heteroelements, a C₆-C₁₂ aralkyl group optionally comprising one or more heteroelements, OH, a group OR⁷, NH₂, a group NR⁷R⁸, SH, a group SR⁷; and R⁷ and R⁸ are chosen, independently of each other, from the following list: a C₁-C₁₂ alkyl group, a C₂-C₁₂ alkenyl group, a C₆-C₁₂ aryl group, a C₆-C₁₂ aralkyl group; the heteroelement(s) being chosen from: Cl, Br, O, S, NR″′, PR″′; and R″′ represents a group chosen from: H, a C₁-C₁₂ alkyl group, a C₂-C₁₂ alkenyl group, a C₆-C₁₂ aryl group and a C₆-C₁₂ aralkyl group,
→C⁺A⁻ represents a task-specific onium salt,
when x=1, C⁺A⁻ may be bonded to Z either via the anion A⁻ or via the cation C⁺,
when x=2, the anion A⁻ and the cation C⁺ are each bonded to a group Z, the two groups 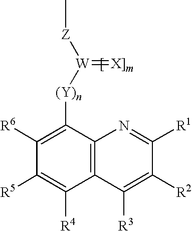
being identical or different,
and at least one solvent, chosen from hydrophobic room-temperature ionic liquid matrices.

7. Composition according to claim 6, in which at least one solvent is chosen from: N,N-dialkylpyrrolidinium, N,N-dialkylpiperidinium and N,N-trialkylammonium.

8. Process for extracting metals in an aqueous composition, this process comprising at least one step of placing in contact the aqueous composition in which the metal(s) is (are) present with a compound of formula (I): 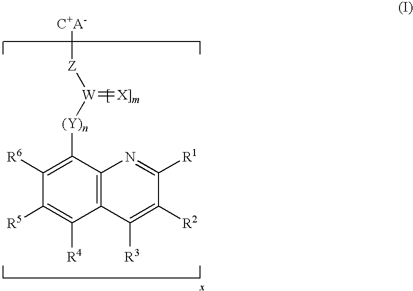
in which:
X represents a group chosen from: O, NR, S, PR, Se; and R represents a group chosen from: H, a C₁-C₁₂ alkyl group optionally comprising one or more heteroatoms, a C₂-C₁₂ alkenyl group optionally comprising one or more heteroatoms, a C₆-C₁₂ aryl group optionally comprising one or more heteroatoms, a C₆-C₁₂ aralkyl group optionally comprising one or more heteroatoms;
Y represents a group chosen from: O, S, NR′, PR′, CR′R″, a C₁-C₁₂ alkanediyl group optionally comprising one or more heteroatoms, a C₂-C₁₂ alkenediyl group optionally comprising one or more heteroatoms, a C₆-C₁₂ aryl group optionally comprising one or more heteroatoms, a C₆-C₁₂ aralkanediyl group optionally comprising one or more heteroatoms; and R′ and R″, which may be identical or different, represent a group chosen from: H, a C₁-C₁₂ alkyl group optionally comprising one or more heteroatoms, a C₂-C₁₂ alkenyl group optionally comprising one or more heteroatoms, a C₆-C₁₂ aralkyl group optionally comprising one or more heteroatoms, a C₆-C₁₂ aryl group optionally comprising one or more heteroatoms;
n is an integer chosen from: 0, 1;
m is an integer chosen from: 1, 2;
x is an integer chosen from: 1 and 2;
W represents an atom chosen from: C and S;
Z represents a group chosen from: a C₁-C₁₂ alkanediyl group optionally comprising one or more heteroelements, a C₂-C₁₂ alkenediyl group optionally comprising one or more heteroelements, a C₆-C₂₀ aralkanediyl group optionally comprising one or more heteroelements, a C₆-C₁₂ aryl group optionally comprising one or more heteroelements, the heteroelement(s) being chosen from: Cl, Br, O, S, NR″′, PR″′; and R″′ represents a group chosen from: H, a C₁-C₁₂ alkyl group, a C₂-C₁₂ alkenyl group, a C₆-C₁₂ aryl group and a C₆-C₁₂ aralkyl group;
R¹, R², R³, R⁴, R⁵ and R⁶, which may be identical or different, each represent a group chosen from the following list: H, Cl, Br, a C₁-C₁₂ alkyl group optionally comprising one or more heteroelements, a C₂-C₁₂ alkenyl group optionally comprising one or more heteroelements, a C₆-C₁₂ aryl group optionally comprising one or more heteroelements, a C₆-C₁₂ aralkyl group optionally comprising one or more heteroelements, OH, a group OR⁷, NH₂, a group NR⁷R⁸, SH, a group SR⁷; and R⁷ and R⁸ are chosen, independently of each other, from the following list: a C₁-C₁₂ alkyl group, a C₂-C₁₂ alkenyl group, a C₆-C₁₂ aryl group, a C₆-C₁₂ aralkyl group; the heteroelement(s) being chosen from: Cl, Br, O, S, NR″′, PR″′; and R″′ represents a group chosen from: H, a C₁-C₁₂ alkyl group, a C₂-C₁₂ alkenyl group, a C₆-C₁₂ aryl group and a C₆-C₁₂ aralkyl group,
→C⁺A⁻ represents a task-specific onium salt,
when x=1, C⁺A⁻ may be bonded to Z either via the anion A⁻ or via the cation C⁺,
when x=2, the anion A⁻ and the cation C⁺ are each bonded to a group Z, the two groups 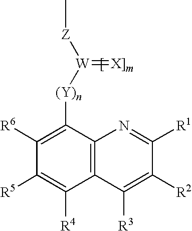
being identical or different.

9. Process according to claim 8, which is performed in a microfluidic device.

10. Process according to either of claims 8 and 9, in which A⁻ represents a group chosen from:
hexafluorophosphate (PF₆⁻), bis(trifluoromethanesulfon-amide) (NTf₂⁻), tris(pentafluoroethyl)trifluorophosphate (FAP), triflate (TfO⁻) and nonaflate (NfO⁻).

11. Process according to any one of claims 8 to 10, in which C⁺A⁻ represents a compound chosen from ammonium, phosphonium and sulfonium salts and also salts resulting from the quaternization of an amine, a phosphine, a thioether or a heterocycle containing one or more heteroatoms such as S, N or P.

12. Process according to any one of claims 8 to 11, in which C⁺ is chosen from ammonium, pyrrolidinium, phosphonium, imidazolium, pyridinium, piperidinium and guanidinium ions.

13. Process for detecting metals in an aqueous composition, this process comprising at least one step according to one of claims 8 to 12 and at least one step of fluorescence measurement.

14. Kit for detecting metals in aqueous medium, comprising a microfluidic device and at least one compound of formula (I) defined in any one of claims 8 to 12.

15. Kit for detecting metals in aqueous medium, comprising at least one compound of formula (I) as defined in any one of claims 8 to 12 and a fluorescence detector.
